(19) United States
(12) Patent Application Publication
(10) Pub. No.: US 2007/0100381 A1
(43) Pub. Date: May 3, 2007

(54) Compensation for cardiac shunt currents during defibrillation

(76) Inventors: David Snyder, Bainbridge Island, WA (US); Daniel Powers, Issaquah, WA (US); Carl Morgan, Bainbridge Is., WA (US)

Correspondence Address: PHILIPS INTELLECTUAL PROPERTY & STANDARDS, P.O. BOX 3001, BRIARCLIFF MANOR, NY, 10510, US

(21) Appl. No.: 10/561,336

(22) PCT Filed: Jun. 1, 2004
(86) PCT No.: PCT/IB04/01939

§ 371 Date: Dec. 15, 2005

Related U.S. Application Data
(60) Provisional application No. 60/483,195, filed on Jun. 27, 2003.

(51) Int. Cl. A61N 1/00

(52) U.S. Cl. 607/5

(57) ABSTRACT

A defibrillator having a pair of electrodes for delivering a defibrillation shock and a method thereof is provided. The defibrillator includes an energy-source circuit that may be discharged through electrodes on a patient to provide a biphasic voltage or current pulse. The energy-source-storage circuit is coupled across a bridge switch for delivering a defibrillation pulse to the patient through a pair of electrodes. A controller operates to control the entire defibrillation process and detects shockable rhythms from the patient via an ECG front end. The controller determines the source of the defibrillator to match the selected mode, which is inputted by connecting coded accessories, such as internal paddles, adult electrodes, or pediatric external electrodes to deliver appropriate defibrillation shocks. Other types of patient-dependent parameters are also employed to achieve the impedance compensation.

### BACKGROUND INFORMATION

1. Field of the Invention

The present invention relates to equipment used in the electrical treatment and monitoring of human bodies and, in particular, to an external defibrillator/cardioverter for optimizing energy delivery to a patient's heart.

2. Description of the Related Art

Electro-chemical activity within a human heart normally causes the heart-muscle fibers to contract and relax in a synchronized manner that results in the effective pumping of blood from the ventricles to the body's vital organs. In a cardiac arrest, a patient is stricken with a life-threatening interruption to his normal heart rhythm, typically in the form of ventricular fibrillation (VF) or ventricular tachycardia (VT) that is not accompanied by a palpable pulse. The only effective treatment for VF is electrical defibrillation, in which an electrical shock is applied to the heart to allow the heart to re-synchronize itself. In a similar manner, non life-threatening arrhythmias such as atrial tachycardia are also treated with electrical shocks in a process often referred to as cardioversion.

External defibrillators/cardioverters (hereafter referred to as defibrillators for brevity) are available to transmit electrical pulses to the patient's heart through electrodes applied to the patient's torso. During the treatment, an operator applies a pair of electrodes across the patient's chest in order to acquire an ECG signal from the patient's heart. If the defibrillator is automated, the defibrillator then analyzes the ECG signal to detect ventricular fibrillation (VF). If VF or other shockable rhythm is detected, the defibrillator delivers a series of defibrillation pulses to resuscitate the patient. The amount of patient current and delivered energy that is required for effective defibrillation depends upon the impedance of the patient. A patient typically has a transthoracic impedance (“patient impedance”) that spans a range commonly understood to be approximately 20 to 180 ohms. In patients, electrical impedance is almost entirely resistive in nature. Thus, it is desirable that a defibrillator provides an impedance-compensated defibrillation pulse that delivers a desired amount of current or energy to any patient' heart across the range of patient impedances.

Although there are many defibrillators that offer impedance compensation, designs for prior-art external defibrillators have not fully addressed cardiac shunt currents. Currents entering the chest from an externally applied defibrillator pad (or paddle) follow myriad pathways through the thorax to re-enter the other defibrillator pad. Some pathways usefully transit the heart, delivering therapeutic current. Other current elements do not transit the heart; these shunt currents are not useful for defibrillation. Nevertheless, these thoracic shunt currents account for the majority of current delivered by the defibrillator. It has been observed that the impedance of shunt current pathways dominates (Deale O C, Lerman, B B, Intrathoracic current flow during transthoracic defibrillation in dogs. Circulation Research 1990, Vol 67, No 6: 1405-1419) total transthoracic impedance and that shunt impedance variation is the likely cause of the wide range observed in human transthoracic impedance.

However, defibrillators are used in a number of delivery modes. For example, a defibrillator may be applied with an anterior-anterior pad position on the patient's chest, so that shunt currents are a major factor as noted above. This delivery mode requires a therapy method providing appropriate compensation for shunt currents. Nevertheless, the same equipment may also be applied in a different delivery mode, such as internal defibrillation using paddles applied directly to the heart. In that mode, shunt currents can be much smaller, so appropriate compensation may be quite different. Further, other delivery modes, including differing external paddle placements or the use of the defibrillator on children, may require other adjustments to the compensation method. Accordingly, there is a need for an improved defibrillation or cardioversion system capable of delivering appropriate impedance-compensated pulses for various intrathoracic-current distributions that may be encountered during the administration of electrotherapeutic pulses for defibrillation and cardioversion for different modes of operation.

### SUMMARY OF THE INVENTION

The present invention is directed to a method and system for providing appropriate defibrillation shocks to a victim of sudden cardiac arrest. The present invention solves the problem of the prior art by providing a defibrillator/cardioverter capable of delivering an impedance-compensated defibrillation pulse that also compensates various degrees of cardiac-shunt currents according to the delivery mode of the defibrillator. An energy-storage circuit, or energy source, is charged to a high voltage by a charger circuit that receives its energy from a battery. The energy-storage circuit is coupled across a mode switch and a bridge switch for delivering a defibrillation pulse to a patient circuit element. The patient circuit element includes a circuit formed by the patient, the particular type of electrodes applied to the patient, and any impedance that may be inherent in the particular electrodes. A controller operates to control the entire defibrillation process and detects shockable rhythms and a defibrillation shock delivery mode via a sensor.

According to one aspect of the invention, a defibrillator includes a pair of electrodes for coupling to a patient; a mode switch having a plurality of output paths coupled to the pair of electrodes, wherein a first output path approximates a voltage source, a second output path acts as a first modified current source, and a third output path acts as a second modified current source; an energy-supply circuit across the mode switch for delivering a defibrillation pulse through the mode switch to the patient; and, a controller coupled to the mode switch and the energy-storage circuit for determining which output path to connect to the patient circuit element depending upon the delivery mode of the defibrillator. In the embodiment, the output of the first output path is substantially equal to the voltage of the energy-supply circuit, the output of the second output path is approximately the voltage of the energy-supply circuit divided by a first impedance plus the impedance presented by the patient, and the output of the third output path is approximately the voltage of the energy-supply circuit divided by a second impedance plus the impedance presented by the patient.

According to another aspect of the invention, a defibrillator is described which comprises a mode switch and an energy source. Both the mode switch position and the energy source voltage depend on a sensed delivery mode.

According to another aspect of the invention, a defibrillator/cardioverter as previously described further adjusts mode switch position and/or energy source voltage based on the success of an earlier delivered electrotherapy.

Another aspect is that the present invention may be realized in a simple, reliable, and inexpensive implementation.

The foregoing and other features and advantages of the invention will be apparent from the following, more detailed description of preferred embodiments as illustrated in the accompanying drawings in which reference characters refer to the same parts throughout the various views. The drawings are not necessarily made to scale; the emphasis instead is placed upon illustrating the principles of the invention.

### DETAILED DESCRIPTION OF THE DRAWINGS

A more complete understanding of the method and apparatus of the present invention is available by reference to the following detailed description when taken in conjunction with the accompanying drawings wherein:

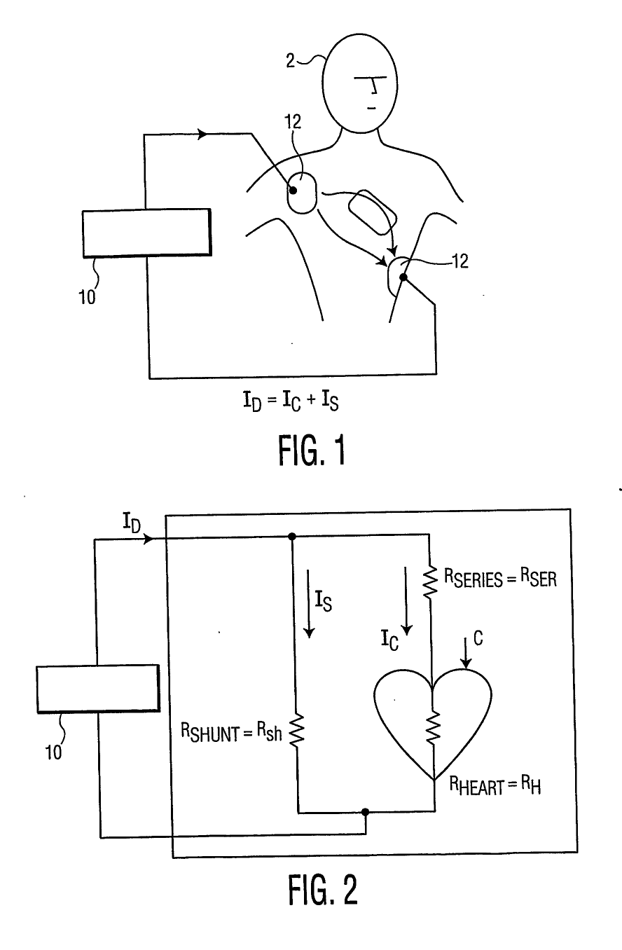

FIG. 1 is an illustration of a defibrillator being applied to a patient under cardiac arrest according to an embodiment of the present invention;

FIG. 2 depicts an equivalent circuit hardware of the defibrillator illustrated in FIG. 1.

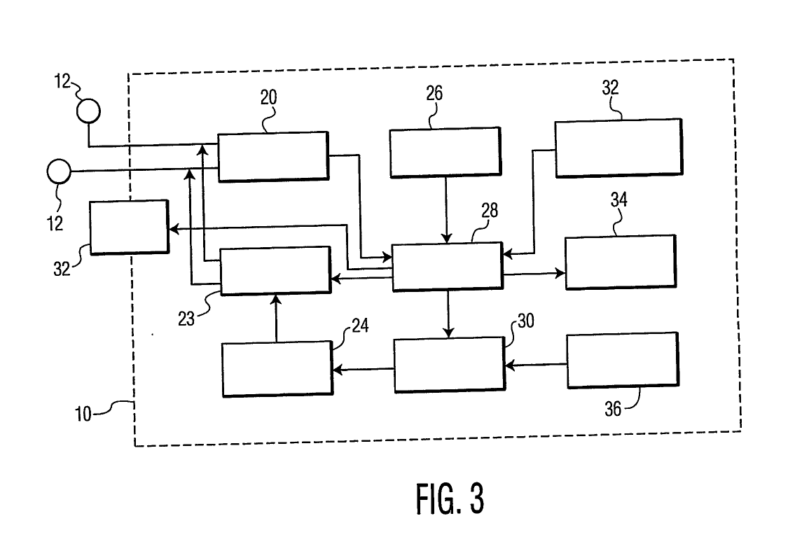

FIG. 3 is a block diagram of a defibrillator system according to a preferred embodiment of the present invention;

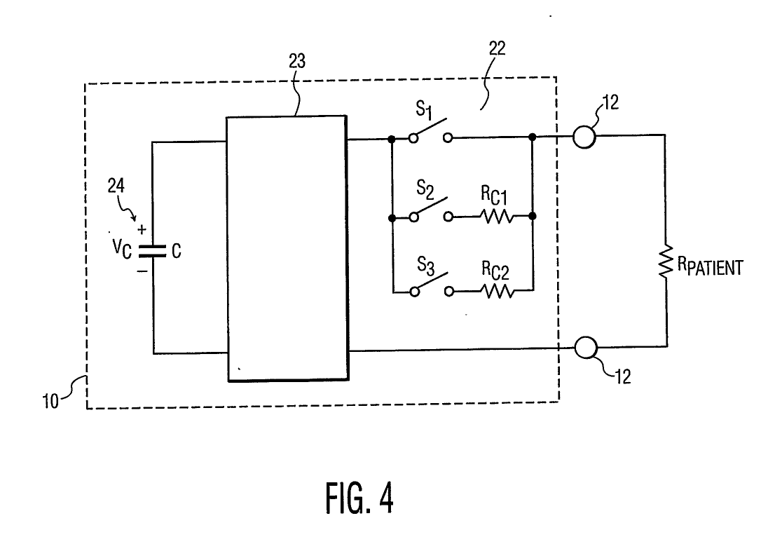

FIG. 4 is a schematic circuit diagram of a defibrillator system according to a preferred embodiment of the present invention; and,

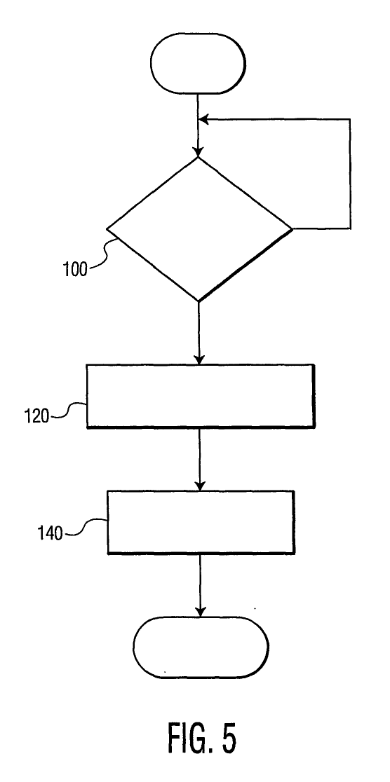

FIG. 5 is a flow chart illustrating the operation steps of a defibrillation system according to a preferred embodiment of the present invention.

### DETAILED DESCRIPTION OF THE EMBODIMENTS

In the following description, for purposes of explanation rather than limitation, specific details are set forth such as the particular architecture, interfaces, techniques, etc., in order to provide a thorough understanding of the present invention. For purposes of simplicity and clarity, detailed descriptions of well-known devices, circuits, and methods are omitted so as not to obscure the description of the present invention with unnecessary is detail.

Methods of delivering defibrillation or cardioversion shocks vary. Pads or paddles may be applied transthoracically in either an anterior-anterior (across the chest) position, or anterior-posterior (chest to back). Patients may range from large adults to small children, with a full range or transthoracic patient impedances. Further, defibrillation may be applied during surgery directly to the heart using internal defibrillation paddles. Each of these methods may require operating the defibrillator in different modes. These different methods of applying the defibrillator shocks to a patient may significantly vary the shunt component of delivered current. Thus, according to the teachings of the present invention, a differing current-distribution method within the patient's body is realized by adjusting the electrical characterstics of a defibrillator to optimize transcardiac current for different defibrillation modes. In particular, selectively changing the source characteristics of a defibrillator to either a modified current source or a voltage source in accordance with the patient's impedance and other information is provided to optimize energy delivery for changes in delivery mode, as explained hereinafter.

Referring to FIG. 1, a defibrillator 10 is configured to deliver a total current, I_{D}, to a patient 2. However, during a delivery of defibrillation shocks in the patient's chest, the current either flows usefully through the patient's heart I_{c}), or shunts around the heart (Iₛ).

FIG. 2 illustrates an equivalent circuit of the defibrillator 10. As shown, the defibrillator 10 outputs currents into either the heart (Rₕₑₐᵣₜ) and series tissues (Rₛₑᵣ) or shunts the heart through the pathway Rₛₕᵤₙₜ.

Prior-art external defibrillators have provided little guidance for the design of an external defibrillator that will approximate a modified current source during shock delivery in order to assure an appropriate cardiac current that is neither too high nor too low. In this regard, we have determined that, in addition to the patient impedance, the choice of delivery methods affects defibrillation or cardioversion success rates across a wide population of patients.

In the case of transthoracic adult defibrillation with anterior-anterior pad or paddle placement, Rₛₕᵤₙₜ may vary considerably, even though the impedance of the path through the heart may exhibit little variability. Thus, an energy supply circuit that approximates a voltage source will supply appropriate cardiac current, shown as Ic in FIG. 2, regardless of the current requirements of the parallel shunt pathway, Is.

In the case of transthoracic defibrillation using anterior-posterior (A-P) pad or paddle application, shunt currents may be a smaller fraction of total current than in the anterior-anterior (A-A) case. Thus, it may be more appropriate to use a modified current source in order to better regulate cardiac current.

In the case of direct internal application for use in surgery to directly defibrillate the heart, Rₛₕᵤₙₜ is very high and Rₛₑᵣ and Rₕₑₐᵣₜ are relatively low. In this case, low-energy current-limited defibrillation pulses are required, as from a modified current source.

In the case of anterior-posterior pediatric application for very small children, Rₛₕᵤₙₜ and Rₛₑᵣ may tend to be lower and a larger fraction of total current is likely to flow through the heart. In this case, a reasonable control of the current source through the heart is needed to insure against excessive cardiac currents.

The present invention is a defibrillator and defibrillation method that takes advantage of the above relationship between the delivery mode and total current and energy delivered to patients. To this end, the defibrillator according to the present invention switches the source characteristics of the defibrillator to match the selected delivery mode, which is inputted by connecting coded accessories, such as internal paddles, adult electrodes, or pediatric-external electrodes; or by an operator-control panel. Further, additional mode information such as patient impedance derived from the patient may be considered when switching the source characteristics of the defibrillator. For example, the defibrillator may sense that adult electrodes are being used in the A-A mode, and that the patient is of relatively high- or low-transthoracic impedance. Based on this patient information, the defibrillator according to the present invention configures its energy-delivery circuitry to approximate a voltage source, or modified current source with one of several series impedances. One defibrillator which can recognize coded accessories is described in U.S. Pat. No. 6,560,485, entitled “Four Contact Identification Defibrillator Electrode System”, hereby incorporated herein.

FIG. 3 is a schematic block diagram of a defibrillator 10 according to a preferred embodiment of this invention. A pair of electrodes 12 for coupling to the patient 2 are connected to a sensor 20 and further connected to a mode switch 22. The sensor 20 operates to detect the type of electrodes, i.e., internal paddles pediatric A-A or A-P electrodes, and adult A-A or A-P electrodes coupled to the defibrillator 10. The sensor 20 may be integrated with an ECG front end, which provides for detection, filtering, and digitizing of the ECG signal from the patient 2. The sensor 20 may optionally sense electrode type independently, as through an identifying coded label on the electrode.

The ECG signal is in turn provided to a controller 28 which runs a shock-advisory algorithm that is capable of detecting ventricular fibrillation (VF) or other shockable rhythm that is susceptible to treatment, by electrotherapy. The ECG front end is also capable of measuring the patient impedance across the electrodes 12 using a low-level test signal that is a non-therapeutic pulse to measure the voltage drop across the electrodes 12. The detected patient impedance is analyzed by the controller 28 to determine the appropriate timing of the waveform to be delivered to the patient. An example of a defibrillator which detects patient impedance is described in U.S. Pat. No. 5,749,904, entitled “Electrotherapy Method Utilizing Patient Dependent Electrical Parameters”, incorporated in its entirety herein. Alternatively, patient impedance may be detected prior to the electrotherapy shock.

A timer 26 is connected to the controller 28 for providing a defibrillation-pulse interval or duration when delivering the defibrillation pulse across the electrode pair 12. A shock button 32, typically part of a user interface of the defibrillator 10, allows the user to initiate the delivery of a defibrillation pulse through the pair of electrodes 12 after the controller 28 has detected VF or other shockable rhythm. It is noted that the activation/deactivation button 32 can function in both AED and manual modes in the preferred embodiment. A display/speaker 34 is connected to the controller 28 and provides audio and visible instructions and/or feedback to the user during the operation of the defibrillator 10.

A battery 36 provides power for the defibrillator 10 in general and in particular for a voltage charger 30 which charges the capacitors in an energy-source circuit 24. Typical battery voltages are 12 volts or less, while the energy-source circuit 24 may be charged to 1800 volts or more. The energy source 24 is a single capacitor or a capacitor bank arranged to act as a single capacitor and can be charged to a range of voltage levels, with the selected level depending on the patient and other parameters. A bridge switch 23 disposed between the energy source 24 and the electrodes 12 controls delivery of energy from the source 24 to the electrodes 12, for example in a multiphasic waveform. A mode switch 22 selectively adds series impedance to the energy delivery circuit as a function of sensed delivery mode. The controller 28 controls the operation of the bridge circuit 23 and the mode switch 22, as well as the voltage charger 30.

During the operation of the defibrillator 10, the controller 28 controls the voltage charger 30 to charge the energy source 24 to a desired voltage level. The initial voltage may be the same for all patients or it may be selected automatically or by the defibrillator user. For example, the defibrillator may have a selection of initial voltage settings, one for an infant, a second for an adult, and a third for use in open chest surgery.

The controller 28 operates the bridge switch 23 to connect energy source 24 with electrodes 12 in one of the two polarities or to disconnect energy source 24 from electrodes 12. The bridge switch 23 selectively connects and disconnects energy source 24 to and from a pair of electrodes 12 electrically attached to a patient 2. Bridge switch 23 operates to deliver the defibrillation pulse across the pair of electrodes 12 to the patient 2 in the desired polarity and duration response to a switch-control signal from the controller 28. The bridge switch 23 is constructed to deliver biphasic-defibrillation pulses in the preferred embodiment but could readily be adapted to deliver monophasic- or multiphasic-defibrillation pulses and still realize the benefits of the present invention.

The controller 28 further operates the mode switch 22 to selectively add appropriate impedance to the electrotherapy delivery circuit, depending on the device operational mode. The operation of the mode switch 22 is described in more detail with reference to FIG. 4.

FIG. 4 depicts a representative circuit diagram of the electrodes 12, energy source 24, bridge switch 23, and mode switch 22 according to a preferred embodiment of the present invention. As shown, the defibrillator 10 comprises an energy-storage capacitor C charged to voltage Vc, a bridge switch 23 for reversing the polarity of the capacitor during discharge, and a mode switch 22 comprised of a series of switches and resistors. It will be apparent to those skilled in the art that other hardware configurations from the one shown can be used successfully.

The controller 28 activates the voltage charger 30 to charge energy-storage capacitor C of the energy source 24 to a predetermined voltage. During this period, bridge switch 23, and optionally switches S1, S2, and S3, are open so that no voltage is applied to the patient connected between electrodes 12. Meanwhile, the controller 28 determines an operation mode according to predetermined criteria (explained later) and adjusts the set of configuration of the mode switch 22 and the energy source 24 voltage prior to delivering the pulse to the patient's heart. Here, the controller 28 can determine the operation mode according to the user's input or by sensing an operational mode parameter, such as coded internal paddles, adult A-A or A-P external electrodes, or pediatric A-A or A-P external electrodes, as sensed by the sensor 20. At the same time, the controller 28 determines the patient impedance via a signal received across the electrodes 12. Then, the adjusted voltage level of the energy source 24 corresponding to the selected operation mode is used to deliver the desired impedance-compensated defibrillation pulse to the patient.

The classification of the operation mode can be made, for example, as follows:

(1) For a defibrillation of adult patients using A-A pads or paddles, a voltage-source application is selected, wherein the capacitor C of the energy source 24 is charged to a voltage that will generate the desired current though the cardiac pathway. During discharge, switch S1 is closed, shorting all series impedance in the output circuit and the capacitor applies its full voltage to the patient. In this case, the term “voltage source” is used as an approximate description of a source with a very low series impedance. A voltage source is capable of supplying a great deal of current while maintaining, for at least short intervals, a predetermined voltage at its terminals. In defibrillators, the initial connection of a charged capacitor with little additional series impedance instantaneously approximates a voltage source quite well.

(2) For an adult defibrillation of low-impedance patients or for an internal or pediatric defibrillation, a modified current-source application is selected, wherein the capacitor C of the energy source 24 may be charged to a higher voltage than the above voltage-source-application mode. During this discharge, switch S2 is closed, placing resistor Rc1 in the patient circuit. Rc1 is configured to present an additional impedance added to the patient and electrode impedance, thus causing the defibrillator to deliver current relatively less affected by patient-impedance variation than in the case of applying a voltage source with little or no additional impedance. In this mode, a “modified current source” is described as a voltage source with additional series impedance. A true current source exhibits infinite impedance and develops whatever voltage at its terminals is necessary to cause a predetermined current to flow from its terminals. This could be approximated with a large series resistance and a very high voltage capacitor, but such an implementation would be very energy-inefficient and impractical. Instead, the term “modified current source” for purposes of this invention refers to a circuit that permits less current variation as a function of load impedance than a voltage source while not wasting a large fraction of stored energy. In the preferred embodiment, the modified current source is a charged capacitor discharged through a selectable resistance. However, in other embodiments the term may refer to other implementations using, for example, inductors to instantaneously moderate current changes.

(3) For an adult defibrillation of A-P patients or pediatric A-A patients, the capacitor C of the energy source 24 is charged to a voltage higher than for the operation mode (1) but not as high as for the operation mode (2). During discharge, switches S2 and S3 are closed such that the parallel combination of Rc1 and Rc2 lowers the source impedance of the defibrillator and does not apply full voltage to the patient. In the embodiment, Rc1 is preferably about 40 ohms and Rc2 is about 20 ohms, so that the ratio between the resistors is about 2 to 1.

Table 1 shows one possible set of mode switch S1, S2, and S3 positions for the delivery modes as discussed above.

TABLE 1

| | Pad or Paddle type indication | | |
|---|---|---|---|
| | delivery mode: | | |
| | S1 State | S2 State | S3 State |
| Adult Anterior—Anterior (A—A) | Closed | Open | Open |
| Adult Anterior-Posterior (A-P) | Open | Closed | Closed |
| Pediatric anterior—anterior (A—A) | Open | Closed | Closed |
| Pediatric Anterior-Posterior (A-P) | Open | Closed | Open |
| Internal | Open | Closed | Open |

FIG. 5 is a flow chart illustrating the operation steps of delivering an artifact-compensated defibrillation shock that also compensates cardiac-shunt currents according to the embodiment of the present invention.

Initially, the defibrillator 10 is deployed by attaching the electrodes 12 to the cardiac victim to analyze a patient-input signal. At the same time, the voltage charger 30 of the defibrillator 10 operates to charge the capacitor C of the energy source 24 to a predetermined percentage of the voltage level in order to deliver a defibrillation shock.

In step 100, the sensor/ECG front end 20 detects a shockable rhythm, i.e., ventricular fibrillation (VF). If no shockable rhythm is detected, the defibrillator 10 continues to detect the ECG information. If a shockable rhythm is detected, the patient impedance is measured by measuring a low-level test signal or delivering a non-therapeutic signal. The detected shockable rhythm is forwarded to the controller 28 of the defibrillator 10.

In step 120, the controller 28 determines the operation mode of delivering pulse to a patient based on the detected impedance of the patient and the type of current distributions expected in the patient's body, as explained earlier. Again, mode information may be inferred via coded accessories, such as internal paddles, pediatric A-A or A-P electrodes, or adult A-A or A-P electrodes; or manually by a user of the defibrillator 10. In an alternative embodiment, the patient-dependent parameter already would have been measured prior to the step 100 using the sensor/ECG front end 20.

In step 140, using the determined operation mode and patient information, the defibrillator 10 configures its energy-delivery circuit by controlling the mode switch 22 as explained with reference to FIG. 4. The controller 28 then begins the process of delivering the defibrillation pulse and functions as a control system to provide for the proper sequence of successive defibrillation shocks at a predetermined interval. Alternatively, the controller 28 may notify the user via the display 34 to press the shock button 32 to actuate manually the delivery of the defibrillation shock to the patient. After the defibrillation shock is discharged to the patient in step 140, the patient's heart is monitored to determine whether a subsequent defibrillation shock is necessary. If so, the above steps may be repeated to deliver the subsequent defibrillation shock. Depending on the success of a delivered shock, successive shocks may adjust switches S1, S2, and S3 or the defibrillator charge voltage to deliver more current to the patient if needed.

Having thus described the preferred embodiment of a system and method for delivering a defibrillation pulse which compensates for cardiac-shunt currents depending on the defibrillation-delivery mode, it should be apparent to those skilled in the art that certain advantages have been achieved. In particular, the present invention implements appropriate impedance-compensation methods for various intrathoracic-current distributions that may be encountered during defibrillation. For example, adult-transthoracic defibrillation, pediatric defibrillation, and internal defibrillation will result in currents that include or shunt the heart to varying degrees. For current distributions that largely include the heart, defibrillation energy is delivered to approximate a modified current source; for current distributions that have considerable pathways shunting the heart, defibrillation energy is delivered to approximate a voltage source; and for intermediate conditions energy is delivered to assure appropriate transcardiac current in the presence of shunt currents.

While the preferred embodiments of the present invention have been illustrated and described, it will be understood by those skilled in the art that various changes and modifications may be made and equivalents may be substituted for elements thereof without departing from the true scope of the present invention. In addition, many modifications may be made to adapt to a particular situation and the teaching of the present invention without departing from the central scope. Therefore, it is intended that the present invention not be limited to the particular embodiment disclosed as the best mode contemplated for carrying out the present invention, but that the present invention include all embodiments falling within the scope of the appended claims.

1. An external defibrillator with a defibrillation energy delivery circuit comprising:
a delivery mode input; and
a mode switch for selectively adding impedance to said energy delivery circuit to compensate for shunt current, the mode switch position based on said delivery mode input.

2. The defibrillator of claim 1 wherein said delivery mode input comprises a parameter indicating the type of an electrode coupled to the defibrillator.

3. The defibrillator of claim 2, wherein said electrode type parameter is selected from the group consisting of adult, pediatric, or internal electrodes.

4. The defibrillator of claim 2, wherein said delivery mode input further comprises a parameter indicating the position of a pair of said electrodes on a patient.

5. The defibrillator of claim 4, wherein said electrode pair position parameter is selected from the group consisting of anterior-anterior, anterior-posterior, and internal positions.

6. The defibrillator of claim 1, wherein said mode switch further comprises a plurality of switches arranged in parallel, the positions of said plurality of switches based on said delivery mode input.

7. The defibrillator of claim 1, further comprising:
an energy source; and
a voltage charger to charge the energy source to a voltage based on the delivery mode input.

8. The defibrillator of claim 1, wherein the position of said mode switch is further based on a parameter indicating the success of a previous defibrillation shock.

9. The defibrillator of claim 1, wherein the position of said mode switch is further based on a measure of patient impedance.

10. A method for compensating defibrillation current for cardiac shunt currents, comprising the steps of:
detecting the mode of delivery of the defibrillation current; and
selectively adding impedance in series with the defibrillation current based on said detecting step.

11. The method of claim 10, wherein said detecting step comprises detecting the type of an electrode which is coupled to the defibrillator.

12. The method of claim 11, wherein said electrode type is selected from the group consisting of adult, pediatric, and internal electrodes.

13. The method of claim 11, wherein said detecting step further comprises detecting a parameter indicating the position of a pair of said electrodes on a patient.

14. The method of claim 13, wherein said electrode pair position parameter is selected from the group consisting of anterior-anterior, anterior-posterior, and internal positions.

15. The method of claim 10, wherein said step of selectively adding impedance further comprises positioning a plurality of switches arranged in parallel based on said detecting step.

16. The method of claim 10, further comprising the step of charging a defibrillation energy source to a voltage based on said detecting step.

17. The method of claim 10, wherein the step of selectively adding impedance is further based on detecting a parameter indicating the success of a previous defibrillation shock.

18. The method of claim 10, wherein the step of selectively adding impedance is further based on a step of measuring patient impedance.

19. An apparatus for delivering electrotherapy in one of a plurality of delivery modes, comprising:
a mode selector; and
an electrotherapy delivery circuit, responsive to the mode selector, which is selectively configured as one of a voltage source or a modified current source, depending upon the delivery mode.

20. A method for delivering electrotherapy in one of a plurality of delivery modes, comprising the step of:
setting a delivery mode; and
selectively configuring an electrotherapy delivery circuit as one of a voltage source or a modified current source as a function of said delivery mode.
